(19) United States
(12) Patent Application Publication
(10) Pub. No.: US 2009/0123933 A1
(43) Pub. Date: May 14, 2009

(54) MICRORNA BIOMARKERS IN LUPUS

(75) Inventor: Nilamadhab Mishra, Winston-Salem, NC (US)

Correspondence Address: MYERS BIGEL SIBLEY & SAJOVEC, PO BOX 37428, RALEIGH, NC, 27627, US

(73) Assignee: Wake Forest University Health Sciences

(21) Appl. No.: 12/269,244

(22) Filed: Nov. 12, 2008

Related U.S. Application Data
(60) Provisional application No. 60/987,251, filed on Nov. 12, 2007.

(51) Int. Cl. C12Q 1/68

(52) U.S. Cl. 435/6

(57) ABSTRACT

The present invention provides methods of screening a subject for systemic lupus erythematosus (SLE), comprising detecting an increase in an amount of one or more markers associated with SLE in a biological sample from the subject, wherein the one or more markers is selected from the group consisting of miR-16-1, miR-16-2, miR-223, let7a-1, let7a-2. let7a-3, let 7c, let7g, and any combination thereof, whereby detection of the increase in the amount of the one or more markers identifies the subject as having SLE. The invention further provides methods of screening a subject for SLE comprising detecting a decrease in miR-95 in a biological sample from the subject, whereby detection of the decrease in the amount of miR-95 identifies the subject as having SLE.

### STATEMENT OF PRIORITY

This application claims the benefit, under 35 U.S.C. §119(e), of U.S. Provisional Application No. 60/987,251; filed Nov. 12, 2007, the entire contents of which are incorporated by reference herein.

### FIELD OF THE INVENTION

The present invention provides methods directed to screening for and diagnosing systemic lupus erythematosus (SLE) using miRNA bioamarkers.

### BACKGROUND OF THE INVENTION

MicroRNAs (miRNAs) are endogenous, nonprotein-coding, single-stranded RNAs of about 22 nucleotides and constitute a novel class of gene regulators which mostly negatively regulate gene expression (Lee et al., PLoS Comput Biol 3:e67 (2007); O'Driscoll, Anticancer Res 26:4271 (2006); Kusenda et al., Biomed Pap Med Fac Univ Palacky Olomouc Czech Repub 150:205 (2006)). Although the first miRNA, lin-4, was discovered in 1993, the presence of miRNAs in vertebrates was confirmed recently in 2001 (O'Driscoll, Anticancer Res 26:4271 (2006)). The miRNAs are the final product of a multistep maturation process that starts with the generation of a transcript, referred to as primary miRNA (pri-miRNA). The pri-miRNA hosts one or more miRNA precursors with a characteristic hairpin structure (Lee et al., PLoS Comput Biol 3:e67 (2007)). Most pri-miRNAs are regular RNA polymerase II transcripts that undergo capping, splicing and polyadenylation. The miRNA precursor hairpins are usually embedded in the introns of their host genes (about 80%), but can be found in exons or across exon-intron boundaries (Kusenda et al., Biomed Pap Med Fac Univ Palacky Olomouc Czech Repub 150:205 (2006)). Among the 439 human miRNAs currently known (although the estimated number of miRNA genes is as high as 1000), 9%, 22%, 29% and 34% are conserved across invertebrates, vertebrates, mammals and primates, respectively, with 5% being specific to humans (Esau and Monia, Adv. Drug Deliv. Rev. 59: 101-114 (2007)).

The biological roles of only a small fraction of identified miRNAs have been elucidated to date. In fact, biological function of this novel class of gene regulators is only just beginning to be understood. Despite the fact that only a small number of the hundreds of identified miRNAs have been characterized, evidence suggests that miRNAs are important regulators for cell growth, differentiation, and apoptosis (Lee et al., PLoS Comput Biol 3:e67 (2007)). Therefore, miRNAs may be important for normal development and physiology. Consequently, dysregulation of miRNA function may lead to human diseases (Perera et al., BioDrugs 21:97 (2007)). Indeed, both basic and clinical studies have demonstrated that miRNAs are aberrantly expressed in diverse cancers (O'Driscoll, Anticancer Res 26:4271 (2006)). Thus, miRNAs may be involved in cell dedifferentiation, growth, and apoptosis as these are all important cellular events in the development of cancer (Esau and Monia,. Adv Drug Deliv. Rev. 59:101-114 (2007); Hammond, Nat Genet 39:582 (2007)).

A recent study of global expression levels of miRNA in various cancers indicates that miRNA expression patterns are generally more useful than messenger RNA (mRNA) profiles to classify cancers (Eis et al. Proc Natl Acad Sci USA 102:3627 (2005)). Until recently, miRNA studies focused largely on cancers. Several studies showed the role of miRNA in homeostasis and function of the immune system of B lymphocytes, T lymphocytes, macrophages, dendritic cells and the heart (Thai et al., Science 316:604 (2007); Rodriguez et al., Science 316:608 (2007); O'Connell et al., Proc Natl Acad Sci USA 104:1604 (2007); Care et al., Nat Med 13:613 (2007); Taganov et al., Proc Natl Acad Sci USA I (2006). For example, miRNA-155 (miR-155) is shown to play a role in regulating T helper cell differentiation and the germinal center reaction to produce an optimal T cell-dependent antibody response (Thai et al., Science 316:604 (2007); Rodriguez et al., Science 316:608 (2007). Transcriptome analysis of microRNA-155-deficient CD4+ T cells identified a wide spectrum of microRNA-155 regulated genes, including cytokines, chemokines, and transcription factors (Thai et al., Science 316:604 (2007); Rodriguez et al., Science 316:608 (2007).

There is increased interest in use of biomarkers in diagnosing and managing SLE (lupus). Recent studies by several investigators demonstrated the presence of organ specific biomarkers in lupus. For example, pro-inflammatory high density lipoprotein, endothelial cells, anti-phospholipid antibody, endothelial protein C receptor, and blood levels of homocysteine are recognized as biomarkers for cardiovascular disease in lupus. Similarly, SLAM family Ly108, IL-8, x-actinin reactive autoantibodies, “editor” auto-antibodies, chemokines and certain proteins in urine can also serve as biomarkers for kidney disease and severity in lupus. Accordingly, the present invention is directed to the use of miRNAs as biomarkers for systemic lupus erythematosus.

### SUMMARY OF THE INVENTION

A first aspect of the present invention provides a method of screening a subject for systemic lupus erythematosus (SLE), comprising detecting an increase in an amount of one or more markers associated with SLE in a biological sample from said subject, wherein said one or more markers is selected from the group consisting of miR-16-1, miR-16-2, miR-223, let7a-1, let7a-2, let7a-3, let7c, let7g, and any combination thereof, whereby detection of the increase of said one or more markers identifies the subject as having SLE.

Another aspect of the invention provides a method of screening a subject for SLE, comprising detecting an increase in an amount of one or more markers associated with SLE in a biological sample from said subject, wherein said one or more markers is selected from the group consisting of miR-16-1, miR-16-2, miR-223, let7a-1, let7a-2, let7a-3, let7c, let7g, and any combination thereof, whereby detection of the increase in the amount of one or more said markers identifies the subject as having an increased risk of developing SLE.

A further aspect of the invention provides a method of diagnosing SLE in a subject, comprising detecting an increase in an amount of one or more markers associated with SLE in a biological sample from said subject, wherein said one or more markers is selected from the group consisting of miR-16-1, miR-16-2, miR-223, let7a-1, let7a-2, let7a-3, let7c, let7g, and any combination thereof, whereby detection of the increase in the amount of said one or more markers diagnoses the subject as having SLE.

A still further aspect of the invention provides a method of screening a subject for SLE, comprising detecting a decrease in an amount of miR-95 in a biological sample from said subject, whereby detection of the decrease in the amount of miR-95 identifies the subject as having SLE.

Additional aspects of the invention provide a method of screening a subject for SLE, comprising detecting a decrease in an amount of miR-95 in a biological sample from said subject, whereby detection of the decrease in the amount of miR-95 identifies the subject as having an increased risk of developing SLE.

Other aspects of the invention provide a method of diagnosing SLE in a subject, comprising detecting a decrease in an amount of miR-95 in a biological sample from said subject, whereby detection of the decrease in the amount of miR-95 diagnoses the subject as having SLE.

Further aspects of the invention provide methods of identifying a subject as having SLE, comprising detecting an increase in an amount of one or more miRNAs in said subject, wherein said one or more miRNAs is selected from the group consisting of miR-16-1, miR-16-2, miR-223, let7a-1, let7a-2, let7a-3, let7c, let7g, and any combination thereof, whereby detection of an increase in said one or more miRNAs identifies the subject as having SLE.

Still further aspects of the invention provide methods of identifying a subject as having an increased risk of developing SLE, comprising detecting an increase in an amount of one or more miRNAs in said subject, wherein said one or more miRNAs is selected from the group consisting of miR-16-1, miR-16-2, miR-223, let7a-1, let7a-2, let7a-3, let7c, let7g, and any combination thereof, whereby detection of the increase in said one or more miRNAs identifies the subject as having an increased risk of developing SLE.

Additional aspects of the invention provide methods of identifying a subject as having SLE, comprising detecting in said subject a decrease in an amount of miR-95, whereby detection of a decrease in the amount of miR-95 identifies the subject as having SLE.

A further aspect of the present invention provides a method of identifying a subject as having an increased risk of developing SLE, comprising detecting in said subject a decrease in an amount of miR-95, whereby detection of a decrease in the amount of miR-95 identifies the subject as having an increased risk of developing SLE.

The foregoing and other aspects of the present invention will now be described in more detail with respect to other embodiments described herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

### BRIEF DESCRIPTION OF THE FIGURES

FIGS. 1A-C illustrate the unsupervised hierarchical clustering of miRNA expression: miRNA profiles of five normal subjects and five lupus patients from peripheral blood mononuclear cells are clustered. A shows miRNAs with greater than 1.5 fold differential expression between the lupus patient samples and the normal samples. B shows miRNAs with greater than 2 fold differential expression between the lupus patient samples and the normal samples. C shows miRNAs with greater than 3 fold differential expression between the lupus patient samples and the normal samples.

### DETAILED DESCRIPTION OF THE INVENTION

As used herein, “a,” “an” or “the” can mean one or more than one. For example, “a” cell can mean a single cell or a multiplicity of cells.

As used herein, “and/or” refers to and encompasses any and all possible combinations of one or more of the associated listed items, as well as the lack of combinations when interpreted in the alternative (“or”).

Further, the term “about,” as used herein when referring to a measurable value such as an amount of a compound or agent of this invention, dose, time, temperature, and the like, is meant to encompass variations of ±20%, ±10%, ±5%, ±1%, ±0.5%, or even ±0.1% of the specified amount.

As used herein, “nucleic acids” encompass both RNA and DNA, including cDNA, genomic DNA, mRNA, synthetic (e.g., chemically synthesized) DNA and chimeras of RNA and DNA. The nucleic acid can be double-stranded or single-stranded. Where single-stranded, the nucleic acid can be a sense strand or an antisense strand. The nucleic acid can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases.

The term “isolated” can refer to a nucleic acid or polypeptide that is substantially free of cellular material, viral material, or culture medium (when produced by recombinant DNA techniques), or chemical precursors or other chemicals (when chemically synthesized). Moreover, an “isolated fragment” is a fragment of a nucleic acid or polypeptide that is not naturally occurring as a fragment and would not be found in the natural state. Furthermore, an “isolated cell” is a cell that has been separated from other components with which it is normally associated in nature. For example, an isolated cell can be a cell in culture medium.

More specifically, an “isolated nucleic acid” is a DNA or RNA that is not immediately contiguous with nucleotide sequences with which it is immediately contiguous (one on the 5′ end and one on the 3′ end) in the naturally occurring genome of the organism from which it is derived. In other embodiments, an isolated nucleic acid includes some or all of the 5′ non-coding (e.g., promoter) sequences that are immediately contiguous to a coding sequence. The term therefore includes, for example, a recombinant DNA that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant DNA that is part of a hybrid nucleic acid encoding an additional polypeptide or peptide sequence.

Much progress has been made over the last several years in developing molecular biomarkers of systemic lupus erythematosus (SLE) using a new generation of molecular technology such as genomics and proteomics. In particular, it has become clear that approximately 22,000 protein-coding transcripts mRNAs can be used to distinguish most SLE patients from healthy controls. Recently, hundreds of small, non-coding microRNA (miRNAs) have been discovered. MicroRNAs represent a purely regulatory, as opposed to structural, process that fine-tunes mRNA expression. The combinatorial nature of nucleotide complementarity permits individual miRNAs to regulate the expression of hundreds of genes by posttranscriptional modification of their cognate messenger RNAs. Therefore, miRNA expression may be a richer source of information for pathogenesis of diseases than messenger RNA profiling and thus holds the promise of translating into practice as a mechanism-based molecular biomarker for preventive, predictive, personalized and participatory (P4) medicine.

Using a microarray analysis of microRNAs, specific micro RNAs have been demonstrated to be differentially expressed in lupus peripheral blood mononuclear cells (PBMCs) as compared with age and sex matched, healthy normal controls. A stringent criteria of three fold differential miRNA expression levels between lupus and healthy samples was used to identify unique patterns of altered miRNA expression. Such patterns provide complex fingerprints that can serve as molecular biomarkers for lupus diagnosis, prognosis, and/or prediction of therapeutic responses.

Thus, the present invention discloses the identification of miRNAs, the expression of which is associated with SLE, and their use as SLE (lupus) biomarker(s). More particularly, the miRNAs as disclosed herein can be used as mechanism based molecular biomarkers for preventative, predictive, personalized, and/or participatory medicine in SLE.

Accordingly, a first aspect of the present invention provides a method of screening a subject for systemic lupus erythematosus (SLE) comprising detecting an increase in an amount of one or more markers associated with SLE in a biological sample from said subject, wherein said one or more markers is selected from the group consisting of miR-16-1, miR-16-2, miR-223, let7a-1, let7a-2, let7a-3, let7c, let7g, and any combination thereof, whereby detection of the increase of said one or more markers identifies the subject as having SLE.

Another aspect of the invention provides a method of screening a subject for SLE comprising detecting an increase in an amount of one or more markers associated with SLE in a biological sample from said subject, wherein said one or more markers is selected from the group consisting of miR-16-1, miR-16-2, miR-223, let7a-1, let7a-2, let7a-3, let7c, let7g, and any combination thereof, whereby detection of the increase in the amount of said one or more markers identifies the subject as having an increased risk (i.e., predisposition) of developing SLE.

A “subject” of this invention includes any animal susceptible to SLE. Such a subject is generally a mammalian subject, including but not limited to human, primate, dog, cat, pig, rabbit, guinea pig, goat, cow, cattle, horse, and the like. Thus, in some embodiments, a subject can be any domestic, commercially or clinically valuable animal including an animal model of SLE. Subjects may be male or female and may be any age including neonate, infant, juvenile, adolescent, adult, and geriatric subjects. In particular embodiments, the subject is a human. A human subject of this invention can be of any gender, race or ethnic group (e.g., Caucasian (white), Asian, African, Negro, black, African American, African European, Hispanic, Mideastern, etc.). In some particular embodiments of the invention, subjects of the invention are Caucasian and/or African-American (black, Negro).

A “subject in need thereof” is a subject known to have, or suspected of having, or at increased risk of developing, SLE. A subject of this invention can also include a subject not previously known or suspected to have SLE or in need of treatment for SLE. A subject of this invention is also a subject known to have or believed to be at risk of developing SLE. Subjects described herein as being at risk of developing SLE are identified by family history, genetic analysis, environmental exposure and/or the onset of early symptoms associated with the disease or disorder described herein.

The symptoms of SLE include, but are not limited to, achy joints/arthralgia, fever of more than 100° F./38° C., arthritis/swollen joints, prolonged or extreme fatigue, skin rashes, anemia, kidney involvement, pain in the chest on deep breathing/pleurisy, buttertly-shaped rash across the cheeks and nose, sun or light sensitivity/photosensitivity, hair loss, blood clotting problems, Raynaud's phenomenon/fingers turning white and/or blue in the cold, seizures, mouth or nose ulcers, and any combination thereof.

As used herein, a biological sample includes, but is not limited to, a tissue sample, whole tissue, a whole organ (e.g., an entire brain, liver, kidney, etc.), bodily fluid sample (e.g., blood, saliva, urine and the like), cell culture, cell lysate, cell extract or the like. In a preferred embodiment, the biological sample comprises or is obtained from a population of cells. By a “population of cells” herein is meant at least two cells, with at least about 10³ cells being preferred, at least about 10⁶ cells being particularly preferred, and at least about 10⁸ to 10⁹ cells being especially preferred. The population or sample can contain a mixture of different cell types from either primary or secondary cultures, and/or from a complex tissue such as a tumor, or may alternatively contain only a single cell type. In one embodiment, peripheral blood mononuclear cells are used. In another embodiment T-cells are used. In still further embodiments of the present invention, CD4 positive (+) T cells are used.

Thus, in some embodiments of the present invention, CD4 positive T cells are isolated for use in the methods described herein. Thus, CD4 positive T cells can be isolated by art-known methods including, but not limited to, negative selection using commercially available antibody and magnetic beads (Miltenyi Biotech CD4 T cell negative isolation kit). These methods are described in further detail in Mishra et al. (J. Immunol. 165:2830 (2000)) and in Mishra et al. (Proc. Natl. Acad. Sci. USA 98:2628 (2001)), the disclosures of which are incorporated by reference in their entireties. Flow cytometry, or any other method known to those of skill in the art, can be used to determine the purity of the isolation and to rule out contamination from CD8 cells, B cells and/or natural killer cells. Flow cytometry can also be used to determine the percent of CD4 T cells having activated, memory or regulatory T cells using antibodies including, but not limited to, anti-CD69, CD45RA, CD70, CD62, CCR7, CD27, CD25, Foxp3 antibodies, any combination thereof, and the like.

In the methods described herein, the detection and quantification of a miRNA marker of this invention in a subject can be carried out according to methods well known in the art as described in the Examples provided herein. For example, RNA is obtained from any suitable sample from the subject that will contain RNA and the RNA is then prepared and analyzed according to well-established protocols for the presence and/or identification of miRNA(s) according to the methods of this invention.

The purified miRNAs are labeled using methods known in the art. Thus, for example, the labeling can be done using a mirVana™ miRNA Labeling Kit (Ambion) and the amine-reactive dyes as recommended by the manufacturer. Amine-modified miRNAs can be cleaned up and coupled to NHS-ester modified Cy5 or Cy3 dyes (Amersham Bioscience). The lupus samples can be labeled with Cy5 and healthy controls will be labeled with Cy3. Unincorporated dyes are removed and the samples hybridized in duplicate according to methods known to those of skill in the art. Thus, for example, the mirVana™ miRNA Bioarrays (Ambion) kit can be used according to the manufacturer's instructions.

A further aspect of the present invention provides a method of diagnosing SLE in a subject comprising detecting an increase in an amount of one or more markers associated with SLE in a biological sample from said subject, wherein said marker is selected from the group consisting of miR-16-1, miR-16-2, miR-223, let7a-1, let7a-2, let7a-3, let7c, let7g, any combination thereof, whereby detection of the increase in the amount of said one or more markers diagnoses the subject as having SLE.

Thus, in some embodiments, an increase in an amount of one or more markers associated with SLE in a biological sample from said subject, wherein said one or more markers is selected from the group consisting of miR-16-1, miR-16-2, miR-223, let7a-1, let7a-2, let7a-3, let7c, let7g, and any combination thereof, comprises an increase over the amount of said one or more markers detected in a biological sample from a normal (control) subject (e.g., a subject that does not have and/or is not suspected of having SLE).

A further aspect of the invention provides a method of screening a subject for SLE, comprising detecting a decrease in miR-95 in a biological sample from said subject, whereby detection of the decrease in the amount of miR-95 identifies the subject as having SLE.

An additional aspect of the invention provides a method of screening a subject for SLE, comprising detecting a decrease in an amount of miR-95 in a biological sample from said subject, whereby detection of the decrease in the amount of miR-95 identifies the subject as having an increased risk of developing SLE.

Other aspects of the invention provide methods of diagnosing SLE in a subject, comprising detecting a decrease in an amount of miR-95 in a biological sample from said subject, whereby detection of the decrease in the amount of miR-95 diagnoses the subject as having SLE.

Thus, in some embodiments, a decrease in an amount of a marker of the present invention, such as miR-95, in a biological sample from said subject, comprises a decrease as compared to the amount of said marker, or miR-95, detected in a biological sample from a normal (control) subject (e.g., a subject that does not have and/or is not suspected of having SLE).

In further embodiments of the invention, a prognostic method is provided. Thus, in some embodiments a method of identifying a subject with SLE having an increased likelihood of a poor prognosis is provided, the method comprising detecting in a subject an increase in an amount of one or more markers associated with a poor prognosis in a population of subjects with SLE, wherein said one or more markers is selected from the group consisting of miR-16-1, miR-16-2, miR-223, let7a-1, let7a-2, let7a-3, let7c, let7g, and any combination thereof, whereby detection of the increase in the amount of said one or more markers identifies said subject as having an increased likelihood of a poor prognosis as compared to a subject diagnosed with SLE in which no increase in the amount of said one or more markers is detected.

In other embodiments of the invention, a method of identifying a subject with SLE having an increased likelihood of a good prognosis is provided, the method comprising detecting in a subject a decrease in an amount of one or more markers associated with a good prognosis in a population of subjects with SLE, wherein said one or more markers is selected from the group consisting of miR-16-1, miR-16-2, miR-223, let7a-1, let7a-2, let7a-3, let7c, let7g, and any combination thereof, whereby detection of the decrease in the amount of said one or more markers identifies said subject as having an increased likelihood of a good prognosis as compared to a subject diagnosed with SLE in which no decrease in the amount of said one or more markers is detected.

In still other embodiments of the invention, a method of identifying a subject with SLE having an increased likelihood of a poor prognosis is provided, the method comprising detecting in a subject a decrease in an amount of miR-95, wherein a decrease in an amount of miR-95 is associated with a poor prognosis in a population of subjects with SLE, whereby detection of the decrease in the amount of miR-95 identifies said subject as having an increased likelihood of a poor prognosis as compared to a subject diagnosed with SLE in which no decrease in the amount of miR-95 is detected.

In further embodiments, a method of identifying a subject with SLE having an increased likelihood of a good prognosis is provided, the method comprising detecting in a subject an increase in an amount of miR-95 associated with a good prognosis in a population of subjects with SLE, whereby detection of the increase in the amount of miR-95 identifies said subject as having an increased likelihood of a good prognosis as compared to a subject diagnosed with SLE in which no decrease in the amount of miR-95 is detected.

Thus, in some embodiments, methods of identifying a subject with SLE having an increased likelihood of a poor prognosis are provided, the methods comprising detecting in a subject a particular miRNA marker profile associated with a poor prognosis in a population of subjects with SLE, wherein said miRNA markers are selected from the group consisting of miR-16-1, miR-16-2, miR-223, let7a-1, let7a-2, let7a-3, let7c, let7g, miR-9 and any combination thereof, whereby detection of the particular miRNA marker profile associated with a poor prognosis identifies said subject as having an increased likelihood of a poor prognosis as compared to a subject diagnosed with SLE in which the same miRNA marker profile is not detected.

In other embodiments, methods of identifying a subject with SLE having an increased likelihood of a good prognosis are provided, the methods comprising detecting in a subject a particular miRNA marker profile associated with a good prognosis in a population of subjects with SLE, wherein said miRNA markers are selected from the group consisting of miR-16-1, miR-16-2, miR-223, let7a-1, let7a-2, let7a-3, let7c, let7g, miR-95 and any combination thereof, whereby detection of the particular miRNA marker profile associated with a good prognosis identifies said subject as having an increased likelihood of a good prognosis as compared to a subject diagnosed with SLE in which the same miRNA marker profile is not detected.

A subject is identified as having SLE according to diagnostic parameters well known in the art and can have a good or poor prognosis according to diagnostic and/or clinical parameters that are also known in the art. For example, a subject with SLE who would be identified as a subject as having a good prognosis is a subject in whom symptoms are mild or moderate, and/or the subject is responsive (i.e., shows improvement) to standard treatment protocols, etc. A subject with SLE who would be identified as having a poor prognosis is a subject in whom symptoms are severe and/or the subject is minimally or non-responsive (i.e., shows minimal to no improvement) to standard treatment protocols. A correlation can be made between good and poor prognosis and a subject's miRNA markers according to the methods of this invention, which can allow a clinician to determine the most effective treatment regimen for the subject. Thus, a poor prognosis or a good prognosis for SLE would be identified by one of ordinary skill in the art.

Accordingly, an association between the likelihood of a poor prognosis and an increase or a decrease in an amount of one or more miRNAs is made by detecting an increase or a decrease in an amount of one or more miRNAs in a population of subjects having SLE and a poor prognosis, i.e., subjects in whom symptoms are severe and/or the subjects are minimally or non-responsive (i.e., shows minimal to no improvement ) to standard treatment protocols; and associating the detected increase or decrease in the amount of the one or more miRNAs with a poor prognosis in the population of subjects having SLE and a poor prognosis.

Similarly, an association between the likelihood of a poor prognosis and a particular miRNA profile is made by detecting an increase or a decrease in an amount of one or more miRNAs in a population of subjects having SLE and a poor prognosis, i.e., subjects in whom symptoms are severe and/or the subjects are minimally or non-responsive (i.e., show minimal to no improvement ) to standard treatment protocols; generating the miRNA profile from the detection of the increase or decrease in the amount of the one or more miRNAs; and associating the miRNA profile with a poor prognosis in the population of subjects having SLE and a poor prognosis.

Alternatively, an association between the likelihood of a good prognosis and an increase or a decrease in an amount of one or more miRNAs is made by detecting an increase or a decrease in an amount one or more miRNAs in a population of patients having SLE and a good prognosis, i.e., subjects in whom symptoms are mild or moderate, and/or the subjects are responsive (i.e., show improvement) to standard treatment protocols; and associating the detected increase or decrease in the amount of the one or more miRNAs with a good prognosis in the population of subjects having SLE and a good prognosis.

Further, an association between the likelihood of a good prognosis and a particular miRNA profile is made by detecting an increase or a decrease in an amount of one or more miRNAs in a population of subjects having SLE and a good prognosis, i.e., subjects in whom symptoms are mild or moderate, and/or the subjects are responsive (i.e., show improvement) to standard treatment protocols; generating the miRNA profile from the detection of the increase or decrease in the amount of the one or more miRNAs and associating the miRNA profile with a good prognosis in the population of subjects having SLE and a good prognosis.

In an additional embodiment, the present invention provides a use of means of detecting in a subject an increase in an amount of one or more markers associated with SLE, wherein said one or more markers is selected from the group consisting of miR-16-1, miR-16-2, miR-223, let7a-1, let7a-2, let7a-3, let7c, let7g, and any combination thereof, whereby detecting an increase in the amount of said one or more markers identifies said subject as having SLE or having an increased risk of developing SLE.

In an additional embodiment, the present invention provides a use of means of detecting in a subject a decrease in an amount of miR-95, whereby detecting a decrease in the amount of miR-95 identifies said subject as having SLE or having an increased risk of developing SLE.

As discussed above, an miRNA of the present invention includes miR-16-1, miR-16-2, miR-223, let7a-1, let7a-2, let7a-3, let7c, let7g, miR-95, and any combination thereof. As used herein, miR-16 can refer to miR-16-1 and/or miR-16-2. In addition, as used herein, let7a can refer to let7a-1, let7a-2, and/or let7a-3. The nucleotide sequences for these miRNAs are known to those of skill in the art (See, e.g., miRNA Database of the The Wellcome Trust Sanger Institute, Wellcome Trust Genome Campus, Cambridge CB10 ISA United Kingdom; Griffiths-Jones et al., Nucleic Acids Research, 2006, Vol. 34, Database issue D140-D144; Griffiths-Jones, S., Nucleic Acids Research, 2004, 32, Database Issue, D109-D111) and are set forth below (Accession numbers are those of the miRNA database of the The Wellcome Trust Sanger Institute, Wellcome Trust Genome Campus, Cambridge CB10 ISA United Kingdom).

(1) miR-16-1 (Accession No. MI0000070)

SEQ ID NO:1

GUCAGCAGUGCCUUAGCAGCACGUAAAUAUUGGCGUUAAGAUUCUAAAAU
UAUCUCCAGUAUUAACUGUGCUGCUGAAGUAAGGUUGAC
(2) miR-16-2 (Accession No. MI0000115)

SEQ ID NO:2

GUUCCACUCUAGCAGCACGUAAAUAUUGGCGUAGUGAAAUAUAUAUUAAA
CACCAAUAUUACUGUGCUGCUUUAGUGUGAC
(3) miR-223 (Accession No. MI0000300)

SEQ ID NO:3

CCUGGCCUCCUGCAGUGCCACGCUCCGUGUAUUUGACAAGCUGAGUUGGA
CACUCCAUGUGGUAGAGUGUCAGUUUGUCAAAUACCCCAAGUGCGGCACA
UGCUUACCAG
(4) let7a-1 (Accession No. MI0000060)

SEQ ID NO:4

UGGGAUGAGGUAGUAGGUUGUAUAGUUUUAGGGUCACACCCACCACUGGG
AGAUAACUAUACAAUCUACUGUCUUUCCUA
(5) let7a-2 (Accession No. MI0000061)

SEQ ID NO:5

AGGUUGAGGUAGUAGGUUGUAUAGUUUAGAAUUACAUCAAGGGAGAUAAC
UGUACAGCCUCCUAGCUUUCCU
(6) let7a-3 (Accession No. MI0000062)

SEQ ID NO:6

GGGUGAGGUAGUAGGUUGUAUAGUUUGGGGCUCUGCCCUGCUAUGGGAUA
ACUAUACAAUCUACUGUCUUUCCU
(7) let7c (Accession No. MI0000064)

SEQ ID NO:7

GCAUCCGGGUUGAGGUAGUAGGUUGUAUGGUUUAGAGUUACACCCUGGGA
GUUAACUGUACAACCUUCUAGCUUUCCUUGGAGC
(8) let7g (Accession No. MI0000433)

SEQ ID NO:8

AGGCUGAGGUAGUAGUUUGUACAGUUUGAGGGUCUAUGAUACCACCCGGU
ACAGGAGAUAACUGUACAGGCCACUGCCUUGCCA
(9) miR-95 (Accession No. MI0000097)

SEQ ID NO:9

AACACAGUGGGCACUCAAUAAAUGUCUGUUGAAUUGAAAUGCGUUACAUU
CAACGGGUAUUUAUUGAGCACCCACUCUGUG

The miRNAs of this invention can be used individually and/or in combination. Thus, in some embodiments, the methods of this invention can include correlations between a particular miRNA, alone or in combination with other miRNAs, and SLE as described herein. For example, the miRNA of this invention, such as miR-95, can be combined with miR-223 in the methods of this invention and in establishing correlations between miRNAs and various aspects of SLE as described herein.

Another aspect of the invention provides methods of identifying a subject as having SLE, comprising detecting an increase in an amount of one or more miRNAs in said subject, wherein said one or more miRNAs is selected from the group consisting of miR-16-1, miR-16-2, miR-223, let7a-1, let7a-2, let7a-3, let7c, let7g, and any combination thereof, whereby detection of an increase in said one or more miRNAs identifies the subject as having SLE.

Other aspects of the invention provide methods of identifying a subject as having an increased risk of developing SLE, comprising detecting an increase in an amount of one or more miRNAs in said subject, wherein said one or more miRNAs is selected from the group consisting of miR-16-1, miR-16-2, miR-223, let7a-1, let7a-2, let7a-3, let7c, let7g, and any combination thereof, whereby detection of an increase in said one or more miRNAs identifies the subject as having an increased risk of developing SLE.

Still other aspects of the invention provide methods of identifying a subject as having SLE, comprising detecting in said subject a decrease in an amount of miR-95, whereby detection of a decrease in said miR-95 identifies the subject as having SLE.

Additional aspects of the invention provide methods of identifying a subject as having an increased risk of developing SLE, comprising detecting in said subject a decrease in an amount of miR-95, whereby detection of a decrease in said miR-95 identifies the subject as having an increased risk of developing SLE.

The miRNAs of this invention are correlated with SLE as described herein according to methods well lnown in the art and as disclosed in the Examples provided herein for correlating miRNAs with various phenotypic traits, including disease states, disorders and pathological conditions and levels of risk associated with developing SLE. Test subjects and control subjects of the present invention are matched by age, gender and/or ethnicity. In general, identifying such correlation involves conducting analyses that establish a statistically significant association and/or a statistically significant correlation between the increase or decrease of a miRNA or a combination of miRNAs and the phenotypic trait in the subject and/or a population of subjects. An analysis that identifies a statistical association (e.g., a significant association) between the miRNA or combination of miRNAs and the phenotype establishes a correlation between the presence of the miRNA or combination of miRNAs in a subject and/or a population of subjects and the particular phenotype being analyzed.

The correlation can involve one or more than one miRNA of this invention (e.g., two, three, four, five, six, seven, eight and/or nine markers or miRNAs) in any combination. In some embodiments of this invention, the miRNAs are miR-16-1, miR-16-2, miR-223, let7a-1, let7a-2, let7a-3, let7c, let7g, miR-95, and any combination thereof, as described above.

Still further aspects of the invention provide methods of identifying an effective treatment regimen for a subject with SLE, comprising correlating an increase in an amount of one or more miRNAs with an effective treatment regimen in a population of subjects with SLE, wherein said one or more miRNAs is selected from the group consisting of miR-16-1, miR-16-2, miR-223, let7a-1, let7a-2, let7a-3, let7c, let7g, and any combination thereof.

Other aspects of the invention provide methods of identifying an effective treatment regimen for a subject with SLE, comprising: a) correlating an increase in an amount of one or more miRNAs in a population of subjects with SLE for whom an effective treatment regimen has been identified, wherein said one or more miRNAs is selected from the group consisting of miR-16-1, miR-16-2, miR-223, let7a-1, let7a-2, let7a-3, let7c, let7g, and any combination thereof, and b) detecting an increase in the amount of one or more markers of step (a) in the subject, thereby identifying an effective treatment regimen for the subject.

Still other aspects of the invention provide methods of correlating an increase in an amount of one or more miRNAs with an effective treatment regimen for SLE, comprising: a) detecting in a population of subjects with SLE and for whom an effective treatment regimen has been identified, an increase in an amount of one or more miRNAs, wherein said one or more miRNAs is selected from the group consisting of miR-16-1, miR-16-2, miR-223, let7a-1, let7a-2, let7a-3, let7c, let7g, and any combination thereof, and b) correlating the increase in the amount of the one or more miRNAs of step (a) with an effective treatment regimen for SLE.

Additional aspects of the invention provide methods of identifying an effective treatment regimen for a subject with SLE, comprising correlating a decrease in an amount of miR-95 with an effective treatment regimen in a population of subjects with SLE.

Other aspects of the invention provide methods of identifying an effective treatment regimen for a subject with SLE, comprising: a) correlating a decrease in an amount of miR-95 in a population of subjects with SLE for whom an effective treatment regimen has been identified; and b) detecting a decrease in the amount of miR-95 of step (a) in the subject, thereby identifying an effective treatment regimen for the subject.

Still other aspects of the invention provide methods of correlating a decrease in an amount of miR-95 with an effective treatment regimen for SLE, comprising: a) detecting in a population of subjects with SLE and for whom an effective treatment regimen has been identified, a decrease in an amount of miR-95; and b) correlating the decrease in the amount of miR-95 of step (a) with an effective treatment regimen for SLE.

Thus, some aspects of the invention provide methods of identifying an effective treatment regimen for a subject with SLE comprising correlating a particular miRNA profile (e.g., an increase or decrease in an amount of one or more miRNAs associate with SLE) with an effective treatment regimen in a population of subjects with SLE, wherein said miRNA profile comprises miRNAs selected from the group consisting of miR-16-1, miR-16-2, miR-223, let7a-1, let7a-2, let7a-3, let7c, let7g, miR-95, and any combination thereof.

Other aspects of the invention provide methods of identifying an effective treatment regimen for a subject with SLE, comprising: a) correlating a particular miRNA profile in a population of subjects with SLE for whom an effective treatment regimen has been identified, wherein said miRNA profile comprises miRNAs selected from the group consisting of miR-16-1, miR-16-2, miR-223, let7a-1, let7a-2, let7a-3, let7c, let7g, miR-95, and any combination thereof; and b) detecting said miRNA profile of step (a) in the subject, thereby identifying an effective treatment regimen for the subject.

Still other aspects of the invention provide methods of correlating a particular miRNA profile with an effective treatment regimen for SLE, comprising: a) detecting in a population of subjects with SLE and for whom an effective treatment regimen has been identified, a particular miRNA profile, wherein said miRNA profile comprises miRNAs selected from the group consisting of miR-16-1, miR-16-2, miR-223, let7a-1, let7a-2, let7a-3, let7c, let7g, miR-95, and any combination thereof, and b) correlating the miRNA profile of step (a) with an effective treatment regimen for SLE.

Examples of standard treatment regimens for SLE are well known in the art and include, but are not limited to, administration of prednisone, hydroxychloroquine, cyclophosphamide, and any combination thereof.

“Treat,” “treating,” or “treatment” refers to any type of action or activity that imparts a modulating effect, which, for example, can be a beneficial effect, to a subject afflicted with a disorder, disease or illness, or at risk of developing a disorder, disease or illness, including improvement in the condition of the subject (e.g., in one or more symptoms), delay in the progression of the condition, prevention or delay of the onset of the disorder, and/or change in clinical parameters, disease or illness, etc., as would be well known in the art.

“Effective amount” refers to an amount of a composition described herein that is sufficient to produce a desired effect, which can be a therapeutic effect. The exact amount of the composition required for an effective amount will vary from subject to subject, depending on the species, age, weight and general condition of the subject, the severity of the condition being treated, the particular composition used, its mode of administration, the duration of the treatment, the nature of any concurrent treatment, the pharmaceutically acceptable carrier used, and like factors within the knowledge and expertise of those skilled in the art. Thus, it is not possible to specify an exact amount for every composition of this invention. However, an effective amount can be determined by one of ordinary skill in the art in any individual case using only routine experimentation given the teachings herein and by reference to the pertinent texts and literature and/or by using routine experimentation. (See, for example, Remington: The Science and Practice of Pharmacy, 21^{st} Edition (2005), Lippincott Williams & Wilkins, Philadelphia, Pa.).

Patients who respond well to particular treatment protocols can be analyzed for a specific miRNA profile (e.g., an increase or decrease in an amount of one or more miRNAs associate with SLE) and a correlation can be established according to the methods provided herein. Alternatively, patients who respond poorly to a particular treatment regimen can also be analyzed for a particular miRNA profile (e.g., an increase or decrease in an amount of one or more miRNAs associate with SLE) correlated with the poor response. Then, a subject who is a candidate for treatment for SLE can be assessed for the presence of the appropriate miRNA profile and the most appropriate treatment regimen can be provided.

Accordingly, an association between an effective treatment regimen and an increase or a decrease in an amount of one or more miRNAs is made by detecting an increase or a decrease in an amount of one or more miRNAs in a population of subjects having SLE and for whom an effective treatment regimen for SLE has been identified; and associating the detected increase or decrease in the amount of the one or more miRNAs with an effective treatment regimen for SLE.

Similarly, an association between an effective treatment regimen and a particular miRNA profile is made by detecting an increase or a decrease in an amount of one or more miRNAs in a population of subjects having SLE and for whom an effective treatment regimen for SLE has been identified; generating the miRNA profile from the detection of the increase or decrease in the amount of the one or more miRNAs; and associating the generated miRNA profile with an effective treatment regimen for SLE.

In some embodiments, the methods of correlating a miRNA profile with treatment regimens can be carried out using a computer database. Thus the present invention provides a computer-assisted method of identifying a proposed treatment for SLE. The method involves the steps of (a) storing a database of biological data for a plurality of patients, the biological data that is being stored including for each of said plurality of patients (i) a treatment type, (ii) at least one miRNA, an increase or decrease in the amount of which is associated with SLE and (iii) at least one disease progression measure for SLE from which treatment efficacy can be determined; and then (b) querying the database to determine the dependence on said increase or decrease in the amount of the at least one miRNA of the effectiveness of a treatment type in treating SLE, to thereby identify a proposed treatment as an effective treatment for a subject having a miRNA profile correlated with SLE.

In one embodiment, treatment information for a patient is entered into the database (through any suitable means such as a window or text interface), miRNA information (e.g., an miRNA profile) for that patient is entered into the database, and disease progression information is entered into the database. These steps are then repeated until the desired number of patients has been entered into the database. The database can then be queried to determine whether a particular treatment is effective for patients having a particular miRNA profile, not effective for patients having a particular miRNA profile, etc. Such querying can be carried out prospectively or retrospectively on the database by any suitable means, but is generally done by statistical analysis in accordance with known techniques, as described herein.

It is further contemplated that the present invention provides kits for use in screening, diagnosing and identifying subjects with SLE, the kits comprising the compositions of this invention (e.g., miRNAs of the present invention, miR-16-1, miR-16-2, miR-223, let7a-1, let7a-2, let7a-3, let7c, let7g, miR-95 and any combination thereof). It would be well understood by one of ordinary skill in the art that the kit of this invention can comprise one or more containers and/or receptacles to hold the reagents (e.g., nucleic acids, and the like) of the kit, along with appropriate buffers and/or diluents and/or other solutions and directions for using the kit, as would be well known in the art. Such kits can further comprise adjuvants and/or other immunostimulatory or immunomodulating agents, as are well known in the art.

The present invention will now be described with reference to the following examples. It should be appreciated that this example is for the purpose of illustrating aspects of the present invention, and does not limit the scope of the invention as defined by the claims.

### EXAMPLES

### Example 1

To determine whether miRNAs play a role in the pathogenesis of human SLE, a novel microarray analysis was performed in peripheral blood mononuclear cells (PBMCs) from human lupus patients and healthy controls. Blood was collected from five lupus patients and seven age and sex matched healthy controls at two different time points over a three month period. The study population included both Caucasian (white) and African American (Negro, black) patients and controls.

The lupus patients had inactive disease and were not on prednisone or cytotoxic agents. Total RNA was isolated from the PBMCs, and miRNAs subsequently isolated with the flashPAGE™ Fractionator system. The lupus samples were labeled with Cy5 and healthy control samples were labeled with Cy3 using the mirVana™ miRNA Labeling Kit. The labeled samples were hybridized to miRNA arrays generated from the mirVana™ miRNA Array Probe Set. Each array experiment was performed in duplicate.

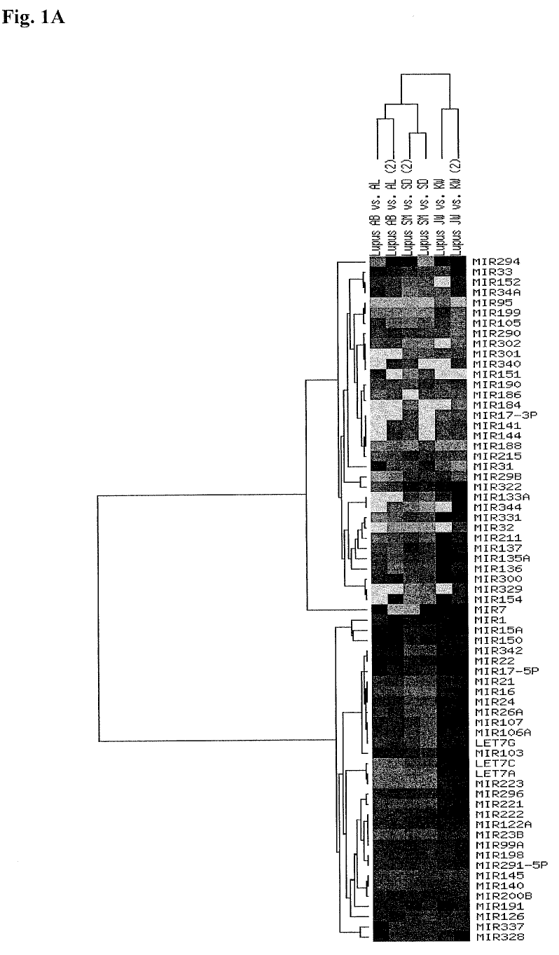

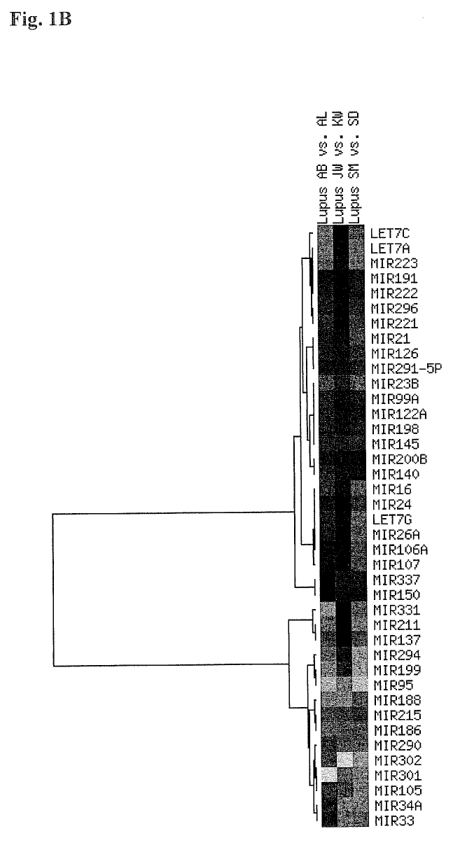

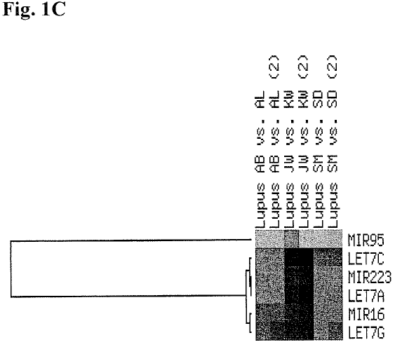

Sixty-seven miRNAs were identified as having a greater than 1.5 fold differential expression and 42 miRNAs were identified with greater than 2 fold differential expression between the lupus samples and normal samples in two experimental pairs (FIGS. 1A and 1B). Correlations between the replicate arrays were 93%, 92% and 96%. Correlations between the 3 different sample pairs were 83%, 94% and 86%.

Six miRNAs were identified as having a greater than 3-fold differential expression between the lupus samples and normal samples (FIG. 1C). Among these six miRNAs, miR-16, miR-223, let7a, let7c, and letg, were increased in lupus patients compared to healthy controls on two different occasions within the three-month period. In contrast, miR-95 expression was decreased in lupus patients compared to healthy controls on two different occasions.

The miRNA target prediction of the differentially expressed miRNAs was performed using a bioinformatics approach. Using four target prediction algorithms (Targetscan, Miranda, Pictar and Sanger), miR 16 was identified as a target for apoptosis. Thus, miR-16 was identified as a target for CDK6, CDC27, CARD10, and/or Bc12. Without wishing to be bound by any particular theory of the invention, it appears that upregulation of miR-16 in lupus patients may inhibit these anti-apoptotic genes, thus allowing the cells to undergo apoptosis. Moreover, by inhibiting these cyclin kinases, miR-16 may inhibit cell cycle progression and accumulate the cell in G0/G1 phase inducing apoptosis. Therefore, these studies indicate that increased expression of miR-16 in peripheral blood mononuclear cells from lupus patients may play a role in the observed aberrant apoptosis in SLE.

Using the same four target prediction algorithms (Targetscan, Miranda, Pictar and Sanger), miR-95 was identified as a target for Toll receptor 6, GATA3, NFAT, MEF2A and/0r SP3. Without wishing to be bound by any particular theory of the invention, it appears that down regulation miR-95 results in increased expression of these transcription factors, thus resulting in aberrant gene expression in lupus patients. In addition, miR 223 was identified in these same studies as a target for histone deacetylase HDAC2, HDAC6 and/or HDAC8.

Thus, in these studies, five miRNAs (miR-16, miR-223, let7a, let7c, let-g) were found to be increased and one miRNA (miR-95) was found to be decreased consistently in lupus patients compared to healthy controls on two different occasions within the three month period in the same individuals. Using four target prediction algorithms (Targetscan, Miranda, Pictar and Sanger), miR-16 was identified as having an important role in apoptosis. Aberrant apoptosis and T cell function are known to play a role in pathogenesis of SLE. Without wishing to be bound by any particular theory of the invention, it appears that selected miRNAs may be over or under expressed in naïve T cells, in the resting state and/or upon activation and that, specific patterns of miRNA expression are responsible for aberrant apoptosis and T cell activation and/or function in SLE.

### Example 2

Expression of miRNAs in Lupus T Cells.

Study Subjects: Sixty adult female and male subjects between the ages of 19 and 65 with a diagnosis of SLE will be recruited from our weekly Lupus Clinic. These subjects fulfill the ACR criteria (≧4 1997 revised ACR (American College of Rheumatology)) for the diagnosis of SLE. Both active and inactive lupus patients will be recruited for the study. Whenever possible, attempts will be made to recruit new patients for whom no medications have been given.

SELENA and SLEDAI criteria will be used for quantifying the level of active or inactive disease states. Studying patients from both inactive and active disease conditions will assist in determining whether aberrant miRNA is a contributory cause or an effect of the disease. The ratio of females to males in our Lupus Clinic is 8.5:1 and the racial composition is approximately 64% white, 34% black, and 2% other. If patients are on prednisone therapy, the dose should be ≦10 mg per day. Blood will be obtained by venipuncture 24 hr after their last dose of prednisone, nonsteroidal anti-inflammatory agents and hydroxychloroquine.

In addition, sixty age, sex and race matched, healthy controls will be recruited and twenty rheumatoid arthritis individuals will be recruited as disease controls. Each subject will sign an IRB-approved consent form prior to participation in the studies.

Statistical Analysis: Approximately 60 subjects/patients will be enrolled in each of the patient and control groups: healthy controls, active lupus and inactive lupus. This maintains 20 subjects or patients for study at one time in order to have a minimum of 15 per group for comparisons among group. Power was calculated assuming a fixed sample size of 14 in each group, a two-sided test and a significance level of alpha=0.05 for effect sizes of 1.0, 1.1 and 1.2 respectively. Thus, the power is 0.72, 0.80, 0.86, respectively, assuming these respective effect sizes, where the choices of effect sizes were made based on preliminary data and a priori hypotheses of influences of patients/subject group.

Effect size is defined as the difference in mean values across groups divided by standard deviation. The difference in mean values specified was 30% across groups, with standard deviations of 30%, 27.5% and 25%, respectively. Although examination of initial data suggests that most of the outcomes of the present investigation will approximately conform to required assumptions for parametric statistical analyses; this will be reassessed after the final data collection and more individuals will be recruited, if needed.

CD4+T cell isolation: The CD4 positive T cells will be isolated by negative selection using commercially available antibody and magnetic beads (Miltenyi Biotech CD4 T cell negative isolation kit). These methods are described in further detail in Mishla et al. (J Immunol 165:2830 (2000)) and in Mishra et al. (Proc Natl Acad Sci USA 98:2628 (2001)), the disclosures of which are incorporated by reference herein in their entireties. The purity of the isolation is 98% as measured by flow cytometry. Flow cytometry will be performed in individual samples to rule out contamination from CD8, B and natural killer cells. As the phenotype of circulating CD4 T cells differ from lupus patients versus control, flow cytometry will be performed to determine the percent of CD4 T cells having activated, memory or regulatory T cells. This will be done using anti-CD69, CD45RA, CD70, CD62, CCR7, CD27, CD25, and/or Foxp3 antibodies.

miRNA isolation: Total RNA isolation and small RNA enrichment procedures will be performed using the mirVana™ miRNA Isolation Kit (Ambion) according to the manufacturer's instructions. To isolate miRNA fractions, total RNA samples will be fractionated and cleaned up with the flashPAGE™ Fractionator and reagents (Ambion) per the manufacturer's recommendation. Briefly, 10 μg of each RNA sample will be loaded onto the top of a column filled with a denaturing acrylamide gel matrix and fractionated by applying an electrical current. A dye will be loaded with the total RNA sample to track RNAs that are about 40 nucleotides in size. Electrophoresis will be stopped when the dye reaches the bottom of the column and miRNAs will be recovered from the bottom buffer chamber using a glass fiber filter-based cleaning procedure (flashPAGE™ Reaction Cleanup Kit, Ambion). Approximately 1 ng of miRNA is recovered per 10 μg of total RNA.

miRNA labeling, cleanup and microarray hybridization and array data processing: Purified miRNAs will be labeled using the mirVana™ miRNA Labeling Kit (Ambion) and amine-reactive dyes as recommended by the manufacturer. The amine-modified miRNAs will be then cleaned up and coupled to NHS-ester modified Cy5 or Cy3 dyes (Amersham Bioscience). The lupus and rheumatoid arthritis samples will be labeled with Cy5 and healthy control samples will be labeled with Cy3. Unincorporated dyes will be removed and the samples will be hybridized in duplicates to mirVana™ miRNA Bioarrays (Ambion) according to the manufacturer's instructions. For each array, the minimum observable threshold will be determined by examining the foreground minus background median intensities for ‘EMPTY’ spots. The minimum threshold will be defined as the 5% symmetric trimmed mean plus 2 standard deviations across all ‘EMPTY’ spots on an individual array.

Data analyses: A one-way analysis of variance (ANOVA) model will be used to test the hypothesis that there is no difference in expression between groups for each miRNA on the array. Pair-wise comparisons for differentially expressed genes identified by ANOVA will be performed to measure relative differences. Two-dimensional unsupervised hierarchical clustering using average linkage and the correlation distance metric will be performed on all the miRNA normalized expression values or on those miRNAs determined to be differentially expressed by one-way ANOVA. Multiple regression analysis will be used to look for effects of additional variables such as drugs and disease activity.

Microarray data validation: qRT-PCR (quantitative real time PCR) reactions will be performed using SuperTaq™ Polymerase (Ambion) and the mirVana™ qRT-PCR miRNA Detection Kit and Primer Sets (Applied Biosystems) following the manufacturer's instructions. qRT-PCR is performed with 5-50 ng of total RNA input on an ABI7500 thermocycler (Applied Biosystems, Foster City, Calif., USA).

Results: Unique expression profiles of miRNAs in active versus inactive lupus patients versus healthy control are expected, similar to that shown in the data in Example 1. A different miRNA expression profile is also expected between the SLE patients and those with rheumatoid arthritis. Such miRNA signatures as determined by the present invention will provide unique mechanistic based biomarkers for lupus that can be used for preventive, predictive, personalized and participatory (P4) medicine.

### Example 3

Determination of the Sensitivity to Apoptosis in T Cells Resulting from Either Increased or Reduced Expression of miR-16.

In contrast to animal models of lupus, lupus T cells exhibit enhanced spontaneous and diminished activation-induced apoptosis and predisposition to necrosis (Gergely et al., Arthritis Rheum 46:175 (2002); Cohen, Springer Semin Immunopathol 28:145 (2006); Emlen et al., J Immunol 152:3685 (1994)). Preliminary data with several microRNAs show that miR-16 is upregulated 3 fold in lupus PBMCs compared to matched healthy controls. Recent studies have demonstrated that miR-16 induces apoptosis by targeting Bc12 and regulates cell cycle progression (Linsley et al., Mol Cell Biol 27:2240 (2007); Cimmino et al., Proc Natl Acad Sci USA 102:13944 (2005)). Moreover, levels of miR-16 are decreased in New Zealand Black mouse (NZB) lymphoid tissue, particularly in the spleen and further, exogenous miR-16 delivered to a NZB malignant B1 cell line results in increased apoptosis (Raveche et al. Blood 109:5079-5086 (2007)). Based upon the preliminary data presented herein studies will be carried out to determine if increased expression of miR-16 in lupus T cells results spontaneous apoptosis.

Material and methods: Isolated CD4 T cells from healthy controls and lupus T cells will be transfected with commercially available siRNA against miRNA16 and with miRIDIAN mimic mir-16 (Dharmacon) using the Amaxa nucleofactor according to manufacturer's protocol. Alternatively, retroviral transduction systems can be used instead of the Amaxa system. Appropriate negative controls from commercial sources will be used in the experiments. T-cells will be plated and incubated at 37° C. for 24 hrs. Following incubation, cells will be stimulated with CD3 and CD28 to induce activation and apoptosis. Flow cytometry will be used to detect cell cycle changes and apoptosis by staining the DNA with propidium iodide using Becton Dickinson FACS Caliber. Acquisition will be done using CELLQUEST™ software and analysis will be performed using ModFit LT™ software. As miR-16 induces apoptosis by affecting Bc12 expression, the levels of Bc12 expression protein will be determined by western blot using Bc12 antibody.

Overexpression of miR-16 is expected to increase apoptosis in normal healthy control T cells whereas depletion of miR-16 by siRNA is expected to decrease apoptosis in lupus T cells. Overexpression of miR 16 is expected to decrease Bc12 expression in healthy control T cells resulting in apoptosis. T cell specific miR-16 deficient mice will be created by a Cre-Lox system to further study the role of miR-16 in lupus pathogenesis. The role of other aberrantly expressed miRNAs in lupus T cells will be tested using similar approaches.

The foregoing is illustrative of the present invention, and is not to be construed as limiting thereof. The invention is defined by the following claims, with equivalents of the claims to be included therein.

All publications, patent applications, patents and other references cited herein are incorporated by reference in their entireties for the teachings relevant to the sentence and/or paragraph in which the reference is presented.

That which is claimed is:

1. A method of screening a subject for systemic lupus erythematosus (SLE), comprising:
detecting an increase in an amount of one or more markers associated with SLE in a biological sample from said subject, wherein said one or more markers is selected from the group consisting of miR-16-1, miR-16-2, miR-223, let7a-1, let7a-2, let7a-3,let7c, let7g, and any combination thereof, whereby detection of the increase in the amount of said one or more markers identifies the subject as having SLE.

2. A method of screening a subject for SLE, comprising:
detecting an increase in an amount of one or more markers associated with SLE in a biological sample from said subject, wherein said one or more markers is selected from the group consisting of miR-16-1, miR-16-2, miR-223, let7a-1, let7a-2, let7a-3, let7c, let7g, and any combination thereof, whereby detection of the increase in the amount of said one or more markers identifies the subject as having an increased risk of developing SLE.

3. A method of diagnosing SLE in a subject, comprising:
detecting an increase in an amount of one or more markers associated with SLE in a biological sample from said subject, wherein said one or more markers is selected from the group consisting of miR-16-1, miR-16-2, miR-223, let7a-1, let7a-2, let7a-3, let7c, let7g, and any combination thereof, whereby detection of the increase in the amount of said one or more markers diagnoses the subject as having SLE.

4. A method of screening a subject for SLE, comprising:
detecting a decrease in an amount of miR-95 in a biological sample from said subject, whereby detection of the decrease in the amount of miR-95 identifies the subject as having SLE.

5. A method of screening a subject for SLE, comprising:
detecting a decrease in an amount of miR-95 in a biological sample from said subject, whereby detection of the decrease in the amount of miR-95 identifies the subject as having an increased risk of developing SLE.

6. A method of diagnosing SLE in a subject, comprising:
detecting a decrease in an amount of miR-95 in a biological sample from said subject, whereby detection of the decrease in the amount of miR-95 diagnoses the subject as having SLE.

7. A method of identifying a subject as having SLE, comprising:
detecting an increase in an amount of one or more miRNAs in said subject, wherein said miRNA is selected from the group consisting of miR-16-1, miR-16-2, miR-223, let7a-1, let7a-2, let7a-3, let7c, let7g, and any combination thereof, whereby detection of an increase in said one or more miRNAs identifies the subject as having SLE.

8. A method of identifying a subject as having an increased risk of developing SLE, comprising:
detecting an increase in an amount of one or more miRNAs in said subject, wherein said miRNA is selected from the group consisting of miR-16-1, miR-16-2, miR-223, let7a-1, let7a-2, let7a-3, let7c, let7g, and any combination thereof, whereby detection of an increase in said one or more miRNAs identifies the subject as having an increased risk of developing SLE.

9. A method of identifying a subject as having SLE, comprising:
detecting in said subject a decrease in an amount of miR-95, whereby detection of a decrease in said miR-95 identifies the subject as having SLE.

10. A method of identifying a subject as having an increased risk of developing SLE, comprising:
detecting in said subject a decrease in an amount of miR-95, whereby detection of a decrease in said miR-95 identifies the subject as having an increased risk of developing SLE.


SEQUENCE LISTING (9 sequences)

SEQ ID NO: 1 - RNA, 89 nt, Homo sapiens
gucagcagugccuuagcagcacguaaauauuggcguuaagauucuaaaauuaucuccaguauuaacugugcugcugaaguaagguugac

SEQ ID NO: 2 - RNA, 81 nt, Homo sapiens
guuccacucuagcagcacguaaauauuggcguagugaaauauauauuaaacaccaauauuacugugcugcuuuagugugac

SEQ ID NO: 3 - RNA, 110 nt, Homo sapiens
ccuggccuccugcagugccacgcuccguguauuugacaagcugaguuggacacuccaugugguagagugucaguuugucaaauaccccaagugcggcacaugcuuaccag

SEQ ID NO: 4 - RNA, 80 nt, Homo sapiens
ugggaugagguaguagguuguauaguuuuagggucacacccaccacugggagauaacuauacaaucuacugucuuuccua

SEQ ID NO: 5 - RNA, 72 nt, Homo sapiens
agguugagguaguagguuguauaguuuagaauuacaucaagggagauaacuguacagccuccuagcuuuccu

SEQ ID NO: 6 - RNA, 74 nt, Homo sapiens
gggugagguaguagguuguauaguuuggggcucugcccugcuaugggauaacuauacaaucuacugucuuuccu

SEQ ID NO: 7 - RNA, 84 nt, Homo sapiens
gcauccggguugagguaguagguuguaugguuuagaguuacacccugggaguuaacuguacaaccuucuagcuuuccuuggagc

SEQ ID NO: 8 - RNA, 84 nt, Homo sapiens
aggcugagguaguaguuuguacaguuugagggucuaugauaccacccgguacaggagauaacuguacaggccacugccuugcca

SEQ ID NO: 9 - RNA, 81 nt, Homo sapiens
aacacagugggcacucaauaaaugucuguugaauugaaaugcguuacauucaacggguauuuauugagcacccacucugug